(19) United States
(12) Patent Application Publication
(10) Pub. No.: US 2006/0124029 A1
(43) Pub. Date: Jun. 15, 2006

(54) Clear paint composition and clear coat film using same

(75) Inventors: Hiroshi Yokoyama, Kanagawa (JP); Masahiko Yamanaka, Kanagawa (JP); Kentarou Watanabe, Kanagawa (JP); Tsunehiko Higuchi, Nagoya (JP); Shigeki Hirabayashi, Nagoya (JP)

Correspondence Address: FOLEY AND LARDNER LLP;SUITE 500, 3000 K STREET NW, WASHINGTON, DC, 20007, US

(73) Assignees: NISSAN MOTOR CO., LTD.; Tsunehiko HIGUCHI

(21) Appl. No.: 11/269,708

(22) Filed: Nov. 9, 2005

(30) Foreign Application Priority Data
Dec. 15, 2004 (JP) 2004-362123

(51) Int. Cl. C08L 89/00; D21H 17/07; C07D 295/18; C08L 97/00

(52) U.S. Cl. 106/124.1; 106/287.25; 106/501.1; 106/316

(57) ABSTRACT

An industrial product is provided with a clear paint composition including a material of a clear paint. An information nucleic acid is contained in the material of the clear paint and includes a site having an arbitrary and known base sequence.

### BACKGROUND OF THE INVENTION

This invention relates to improvements in a clear paint composition and a clear coat film using the clear paint composition, and more particularly to the clear paint composition and clear coat film using an information nucleic acid usable for individuality identification.

In order to identify an individuality of articles, a license plate, a watermarking for paper money, an IC chip, a facial portrait for a credit card and the like have hitherto been employed as individuality identifying means.

However, these individuality identification means have such drawbacks as to be removable from a product, for example, by being peeled off, cut or erased. Accordingly, it has been desired to develop an identification information which cannot be removed or erased from the product.

In this regard, DNA inherently contained in every organism is an information biopolymer containing all genetic information of the organism. Most DNA correspond to many amino acid sequences of protein. DNA includes compounds such as deoxyadenosine (dA), deoxyguanosine (dG), deoxycytosine (dC) and thymidine (dT) which are bonded in a certain direction through phosphoric ester links. Assuming that the number of bases in DNA is n, 4ⁿ kinds of DNA will exist. Accordingly, the existence of about 4.3 billions kinds of distinguishable DNA is assumed even with only 16 kinds of bases. At the present time, in synthesis of DNA having several tens of base sequences, any DNA having any base sequence can be freely synthesized. In addition, concerning DNA in an amount more than a certain level, its base sequence can be automatically determined by an automatic sequence reader or sequencer.

With such a background, the following proposition has been made as disclosed in Japanese Patent Provisional Publication No. 2004-159502 in which a product is provided with a counterfeit-proof label made of a water-insoluble medium containing DNA. The authenticity of the product can be checked according to the presence or absence of DNA.

### SUMMARY OF THE INVENTION

However, the technique disclosed in Japanese Patent Provisional Publication No. 2004-159502 basically relates to a method for mixing DNA with the water-insoluble medium. As a method for checking the authenticity of the product, the publication discloses that an objective product containing ribonucleic acid is identified by detecting whether ribonucleic acid is amplified or not by using a PCR method. Additionally, the publication does not disclose individuality identification data using presence or absence of DNA as an examination index as well as data which relates to individuality identification and makes possible individuality identification of each product even in the same kind of products.

By the way, in case where an article such as a vehicle is stolen or vandalized by an assailant who has run away, it is required to specify an objective article as soon as possible according to the pieces of paint or of the material of the article which pieces have been left at the crime scene.

In view of the above, an object of the present invention is to provide improved clear paint composition and clear coat film which can effectively overcome drawbacks encountered in conventional clear paint compositions and clear coat films.

Another object of the present invention is to provide improved clear paint composition and clear coat film containing an information nucleic acid therein, with which the origins and histories of products can be individually and concretely specified.

An aspect of the present invention resides in a clear paint composition which comprises a material of a clear paint. An information nucleic acid is contained in the material of the clear paint and includes a site having an arbitrary and known base sequence.

Another aspect of the present invention resides in a clear coat film which comprises a solidified clear paint composition including a material of a clear paint. An information nucleic acid is contained in the material of the clear paint and includes of a site having an arbitrary and known base sequence.

### BRIEF DESCRIPTION OF THE DRAWING

FIG. 1A is a structural formula of a natural type DNA;

FIG. 1B is a structural formula in which a hydroxyl group at position 5′ of DNA of FIG. 1A is derivatized;

FIG. 2 is a schematic view showing the base sequence of a single-stranded DNA whose identification information site is provided with primer binding sites at its both ends;

FIG. 3 is a fragmentary sectional illustration showing an example of a laminated coat film including a clear coat film according to the present invention; and

FIG. 4 is a flow chart showing an example of an individuality identification method for the clear paint composition or the clear coat film according to the present invention.

### DETAILED DESCRIPTION OF THE INVENTION

According to the present invention, a clear paint composition comprises a material or raw material of a clear paint, and an information nucleic acid contained in the material of the clear paint and including a site having an arbitrary and known base sequence. With this, the clear paint composition can be easily coated on a product and usable as an excellent individuality identification method since the clear paint composition is difficult to be removed from the product after formation of a coat film of the clear paint composition.

In this specification and claims, all percentages (%) are by mass unless otherwise specified.

The above-mentioned information nucleic acid includes DNA (deoxyribonucleic acid), RNA (ribonucleic acid), and derivatives of DNA and RNA. Although either a natural type nucleic acid or an artificial type nucleic acid can be used, it is preferable to use the artificial type one which is structurally stable, taking account of the information nucleic acid being contained in the clear paint composition used under a severe condition. In the artificial type nucleic acid, an arrangement of bonds whose bonding modes do not exist in the natural type nucleic acid can be formed. In the bonding modes, a bond between nucleoside and nucleoside includes not only a phosphoric ester link but also a nonnatural type one such as a thiophosphoric ester link.

Additionally, concerning the above-mentioned information nucleic acid, “base sequence site is arbitrary” means that a sequence of bases can be freely selected as far as the base sequence is detectable. Further, “the base sequence site has been known” means that the base sequence used for individuality identification has been previously grasped or ascertained.

With respect to size of the information nucleic acid, it is preferable that the number of bases in the whole of the information nucleic acid is not larger than 200. In case where the number of the bases is larger than 200, unreacted sites are made bit by bit at the stage of synthesis so that a content of nucleic acids whose bases are missed is liable to increase. The number of the bases is more preferably about 100.

Further, it is preferable that thymine and thymine are not adjacent to each other in the above base sequence. This prevents dimerization of thymines.

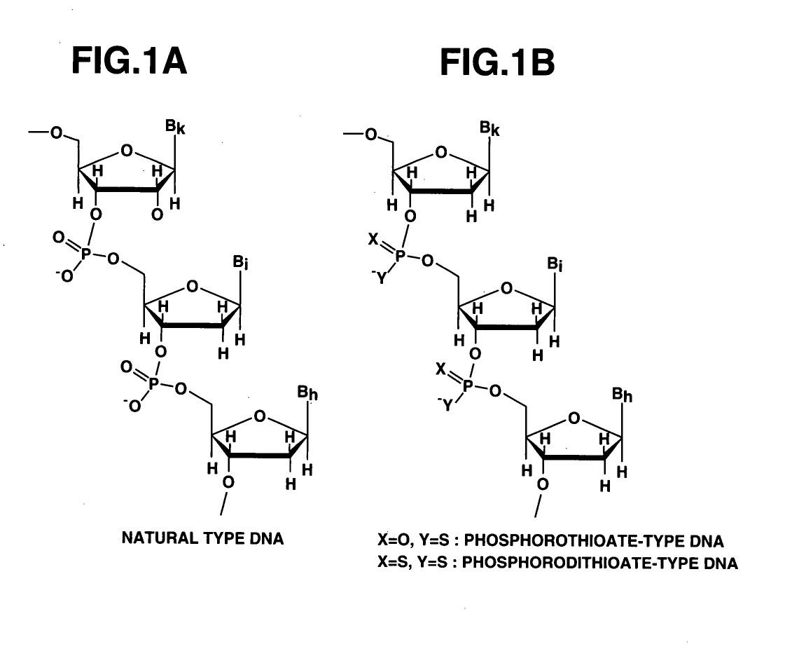

Further, it is preferable that the information nucleic acid is derivatized with a protective group from the viewpoints of improving stability in cases where it is used together with a compound which can react with a hydroxyl group and used under the severe condition. Specifically, at least one of hydroxyl groups at positions 3′ and 5′ can be derivatized with a phosphoric ester group, an acyl group, an alkoxycarbonyl group, a benzyl group, a substituted benzyl group, an allyl group and the like. FIG. 1A shows a structural formula of a natural type DNA, and FIG. 1B shows a structural formula in which a hydroxyl group at position 5′ of DNA generally illustrated in FIG. 1A is derivatized. In FIG. 1B, a generally illustrated DNA is of a phosphorothioate type in case that X is an oxygen atom and Y is a sulfur atom, and the generally illustrated DNA is of a phosphorodithioate type in case that both X and Y are sulfur atoms.

It is further preferable that the hydroxyl group at position 5′ is derivatized with biotin or fluorescent molecules from the viewpoints of improving the convenience in isolation and refinement of the information nucleic acid. Concretely, using biotin to derivative the information nucleic acid facilitates a selective adsorption of the information nucleic acid to a column to which avidin (a kind of protein) is bonded. Meanwhile, using fluorescent molecules such as fluorescein facilitates refinement and the like of the information nucleic acid since nucleic acid itself becomes fluorescent so as to be sensitively detectable. Thus, the improved convenience in isolation and refinement of the information nucleic acid largely facilitates individuality identification.

When RNA is used as the information nucleic acid, it will be understood that a hydroxyl group at position 2′ may be derivatized with the above-mentioned protective groups from the viewpoint of improving stability.

Furthermore, it is preferable that the above base sequence site is a site used for amplification of the information nucleic acid from the viewpoint of achieving an effective detection of the information nucleic acid even in a case that the information nucleic acid in the clear paint composition is low in content. As a method of the above amplification of the information nucleic acid, a polymerase chain reaction (PCR) by which DNA is synergistically amplified may be suitably employed.

Typically, it is preferable to use a PCR method using PCR by which the information nucleic acid even in a very small quantity can be highly amplified. With this PCR method, for example, by acting heat-resistance DNA polymerase on original DNA in the presence of bases or primers complementary to several tens of bases from a terminus or end of the original DNA under a temperature control, the original DNA can be amplified. Therefore, the original DNA can be amplified several hundreds of millions times when this operation for amplification is repeated 30 times. This amplification makes it possible to provide a sufficient amount of DNA to determine the base sequence. As a result, the identity of the product (or the clear paint composition) which had contained the information nucleic acid is authenticated from information that corresponds to the base sequence.

Additionally, in connection with the above, the original DNA preferably has primer binding sites which correspond to primers at its both ends, as the above-mentioned site used for amplification. The information nucleic acid which does not have a primer may be used; however, provision of the primer can make possible distinction of the original DNA within a short time.

Regarding a primer binding site, the number of the bases is preferably not less than 5, and more preferably not less than 10. If the number of the bases is less than 5, the number of nucleic acids which are distinguishable is reduced so that much time is unavoidably spent to individually distinguish a great many objective products (or the clear paint composition) which are intermixed. Meanwhile, the number of the bases is preferably not larger than 100. If the number of the bases is larger than 100, the ratio of by-product lacking for a base at any position is unavoidably increased. Accordingly, it will take much time and effort to refine, or refinement will become difficult to be done in certain circumstances.

When RNA is used as the information nucleic acid, it is understood that first DNA whose base sequence is complementary to the RNA may be produced by using reverse transcriptase, and thereafter the PCR method may be carried out using the thus produced DNA.

Moreover, it is preferable that the information nucleic acid has an identification information site in addition to the above-mentioned base sequence site. With this, more detailed information can be set, thereby accomplishing more advanced individuality identification.

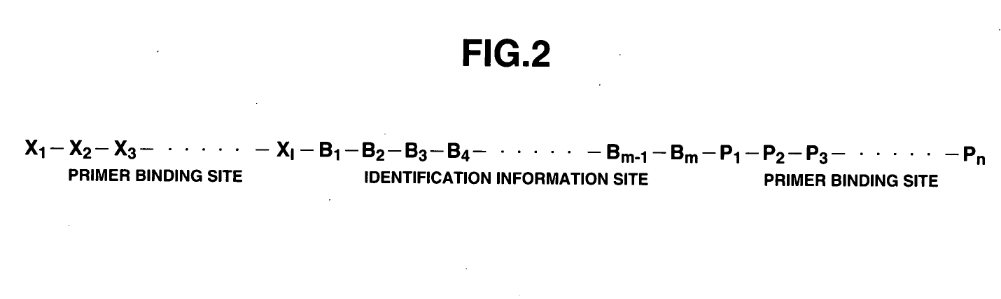

This is, for example, explained with reference to FIG. 2, in which an information DNA having the primer binding sites at its both ends is shown. The information DNA as shown in FIG. 2 has the identification information site (B₁ to Bₘ) which is a base sequence consisting of m (number) bases at the middle. A sequence information of this identification information site (B₁ to Bₘ) corresponds to identification information. The primer binding sites (X₁ to X₁, and P₁ to Pₙ) which are base sequences consisting of 1 (number) and n (number) bases are connected to the both ends of the above identification information site. Each of the primer binding sites is formed complementary to primers having 1 (number) and n (number) bases, respectively. Upon existence of the primer binding sites, use of the PCR method becomes possible for the first time. Either a single-stranded or a double-stranded information DNA can be used as an information element. The double-stranded information DNA is a complex of a first single-stranded information DNA and a second single-stranded information DNA complementary to the first one. The bases in the primer binding site can be sequenced such that bonds between the bases sequenced complementarily to each other can be stable as much as possible and that amplification by the PCR method can progress smoothly.

Further, the information nucleic acid is contained in the material of the clear paint preferably within a range of from 0.5 to 500 μg relative to 100 g of a resinous solid content of the clear paint from the viewpoints of improving an accuracy of detection of the information nucleic acid and a dispersibility of the information nucleic acid in the material of the clear paint. The information nucleic acid is contained more preferably within a range of from 1.0 to 300 μg relative to 100 g of the resinous solid content of the clear paint, and most preferably within a range of from 5.0 to 200 μg relative to 100 g of the resinous solid content of the clear paint. In case that an amount of the content of the information nucleic acid is less than 0.5 μg, it tends to be difficult to distinguish the information nucleic acid from a clear coat film resulting from the clear paint composition. In case that the amount of the content of the information nucleic acid exceeds 500 μg, the coat film tends to lower in transparency when moisture or water contents penetrate to the coat film.

It will be understood that the above resinous solid content of the clear paint means the clear coat film which is formed upon solidification of the clear paint composition.

As the above material of the clear paint, common clear paints, typically those of lipophilic liquid type, hydrophilic liquid type and powder type can be used. The lipophilic liquid type clear paints include an acrylic resin-based paint, a melamine resin-based paint, a urethane resin-based paint and the like. The hydrophilic liquid type clear paints include a hydrophilic acrylic resin-based paint, a hydrophilic melamine resin-based paint, a hydrophilic urethane resin-based paint and the like. The powder type clear paints include a polyester-based powder paint, an acrylic powder paint and the like. The above listed clear paints can be used singly or in combination. The above lipophilic liquid type clear paint may be any of a one package type, two package type (such as a urethane resin paint) and the like. In addition, the clear paint of a lacquer type may be used, and a photocurable resin may be used as the clear paint.

It will be understood that the clear paint composition according to the present invention may suitably contain various additives in addition to the material of the clear paint and the information nucleic acid. The various additives include organic and inorganic pigments, a dispersing agent, a curing accelerator and the like.

Additionally, it is preferable that the information nucleic acid is singly contained in the material of the clear paint and dispersed so as not to make its secondary aggregation or cohesion. This facilitates production of the clear paint composition, while makes it possible to produce the clear paint composition at a low cost. Typical examples of the information nucleic acid are DNA, RNA, β-1,3 glucan. Further, typical examples of the material of the clear paint are lipophilic liquid type paints, hydrophilic liquid type paints, powder type paints and the like. The lipophilic liquid type paints include the acrylic resin-based paint, the melamine resin-based paint, the urethane resin-based paint and the like. The hydrophilic liquid type paints include the hydrophilic acrylic resin-based paint, the hydrophilic melamine resin-based paint, the hydrophilic urethane resin-based paint and the like. The powder type paints include the polyester-based powder paint, the acrylic powder paint and the like. It will be understood that the above-mentioned secondary aggregation means a condition or phenomenon in which particles of the information nucleic acid dispersed in the material of the clear paint aggregate together so as to form larger particles when the clear paint composition is contacted with water.

Further, the information nucleic acid is preferably carried on fine particles. With this, the information nucleic acid contained in the material of the clear paint can be prevented from flowing out of the material of the clear paint, so that a life of the clear paint composition can be prolonged. Additionally, the dispersibility in a solvent is improved so that, for example, the information nucleic acid can be dispersed in the material of the clear paint by using a polar solvent such as methyl ethyl ketone (MEK).

Moreover, it is preferable that the above fine particles have an average particle size ranging from 0.01 to 2 μm from the viewpoints of improving the accuracy of detection of the information nucleic acid and the dispersibility of the information nucleic acid in the material of the clear paint. The fine particles have more preferably an average particle size ranging from 0.02 to 1 μm, and most preferably an average particle size ranging from 0.02 to 5 μm. When the average particle size of the fine particles is smaller than 0.01 μm, the accuracy of detection of the information nucleic acid tends to be lowered. When the average particle size of the fine particles is larger than 2 μm, the coat film is lowered in transparency and smoothness.

Additionally, the content of the fine particles in the material of the clear paint is preferably within a range of from 0.5 to 10% relative to the above resinous solid content of the clear paint from the viewpoints of improving the accuracy of detection of the information nucleic acid and the dispersibility of the information nucleic acid in the material of the clear paint. The fine particles content is more preferably within a range of from 0.5 to 5%, and most preferably within a range of from 1.0 to 5%. When the fine particles content is less than 0.5%, there is a fear that the accuracy of detection of the information nucleic acid is lowered since an amount of the fine particles in a sampled clear coat film is too small. When the fine particles content exceeds 10%, the clear coat film tends to be lowered in transparency.

It is preferable that silica, zinc oxide, titanium oxide, molybdenum oxide, tungsten oxide, barium titanate and/or the like are used as the above fine particles.

In order to carry the information nucleic acid on the above fine particles, first a suspension is prepared by dispersing the fine particles in sterilized distilled water. Thereafter, the above information nucleic acid is directly added to the suspension, thereby obtaining a mixture solution. The mixture solution may be obtained by adding to the suspension an information nucleic acid aqueous solution prepared by mixing the information nucleic acid with sterilized distilled water. Then, the above fine particles are dried to produce fine particles carrying the information nucleic acid. In the above, there is no problem even if a part of the information nucleic acid is directly added to the suspension without forming an aqueous solution while a remaining part is added in the state of an aqueous solution.

Additionally, it is preferable that a solvent(s) is further added to the above suspension. The solvent includes alcohol (such as methanol, ethanol, propanol, butanol, pentanol, hexanol, heptanol, octanol and nonanol), ester (such as ethyl acetate, butyl acetate and propyl acetate), ketone (such as acetone, dimethylketone, methyl ethyl ketone and diethylketone), an aromatic solvent (such as toluene, hexane, cyclohexan and xylene) and the like. With this, the above fine particles are improved in the dispersibility in the suspension while volatilization of water and solvent contents after addition of the information nucleic acid is promoted.

These solvents are not limited to be used singly, and therefore two or more kinds of these solvents may be used in combination. Further, these solvents are allowed to be added at the same time as addition of the information nucleic acid or after addition of the information nucleic acid.

Concerning the amount of the above solvent in case that the solvent is alcohol, a volume ratio of the sterilized distilled water to the alcohol is preferably within a range of from 1 to 99. In case that the solvent is other than alcohol, that is, in case that the solvent is ester, ketone and/or the aromatic solvent, the volume ratio of sterilized distilled water to the solvent is preferably within a range of from 1 to 75.

The above effects of addition of the solvents are not obtained sufficiently if the amount of the solvent is too small. If the amount of the solvent is too large, sufficient effects are apt not to be obtained because compatibility of the solvents with water is lowered so that water cannot volatilize so as to tend to remain.

Hereinafter, the clear coat film resulting from the clear paint composition according to the present invention will be discussed in detail.

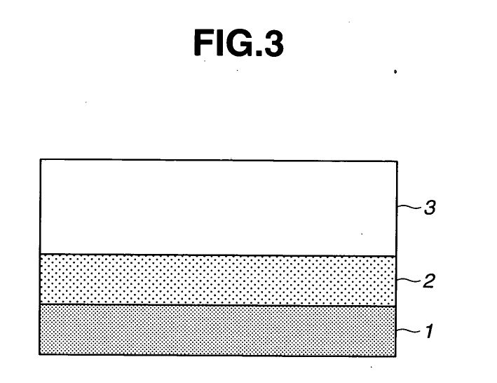

The clear coat film is formed by disposing or coating the above-mentioned clear paint composition on any base material and then by hardening the clear paint composition. As shown in FIG. 3, for example, the clear coat film forms part of a laminated coat film consisting of undercoat film 1, first basecoat layer 2 and clear coat film 3. Clear coat film 3 may contain the information nucleic acid having the primer binding site. In this case, clear coat film 3 containing the information nucleic acid may form a single layer or plural layers.

Further, the above clear coat film may be formed transparent and colorless or transparent and colored so as to be freely colorable. Typically, a transparent and colorless clear coat film can be obtained by singly using the clear paint. The transparent and colored clear coat film can be obtained by using a paint in which various additives such as pigments are contained in such an amount that the paint has a transmittancy of visible light not less than 80% and less than 100%. The above transparent and colored clear coat film includes a so-called turbid clear coat film having a translucent feeling.

Further, although the thickness of the clear coat film is not particularly limited, the thickness is preferably within a range of from about 20 to 40 μm from the viewpoint of improving the accuracy of detection of the information nucleic acid.

Typical examples of the above base material are various metal materials such as iron, aluminum and copper, various organic materials such as polypropylene and polycarbonate, and various inorganic materials such as quartz, ceramics (including calcium carbide and the like). Additionally, commonly known methods may be employed to coat the clear paint composition on these base materials. The commonly known methods include brushing method, spray-coating method, electrostatic-coating method, electrodeposition-coating method, powder coating, sputtering method and the like. Moreover, it is sufficient that the clear coat film is coated in such an extent that the information nucleic acid can be detected, and therefore the clear coat film may be coated on the whole of or a part of the base material.

For the above-mentioned clear paint composition and clear coat film, individuality identification can be accomplished by detecting the information nucleic acid.

In order to determine the base sequence of the information nucleic acid, it is desirable that data of the above information nucleic acid extracted from the clear paint composition or clear coat film is compared with an information nucleic acid database containing at least the data of the above information nucleic acid. Upon comparison with the information nucleic acid database previously grasped or ascertained, a time to be required for identification of the product can be sharply shortened.

Examples of the data stored in the database are a time of electrophoresis, a travel distance of the information nucleic acid during gel filtration (the travel distance can be indicated when the information nucleic acid itself is poured in a control lane), and the like.

Additionally, in the above method of individuality identification, in order to amplify the information nucleic acid by using the PCR method, a solution of the extracted information nucleic acid, a PCR buffer, sterilized distilled water, at least one kind of primer, 2,3-dideoxynucleosidetriphosphate (dNTP) and polymerase are mixed together to obtain a blended solution. Subsequently, the blended solution is heated at 92 to 95° C. for 2 to 5 minutes, as a step (1). Then, the blended solution is subjected to a repeated heat cycle of 20 to 50 cycles each of which consists of first heating (2a) at 92 to 95° C. for 30 to 60 seconds, second heating (2b) at 20 to 50° C. for 30 to 60 seconds, and third heating (2c) at 70 to 80° C. for 30 to 120 seconds. Thereafter, the blended solution is preferably subjected to a heat treatment at 70 to 80° C. for 1 to 10 minutes, as a step (3). It will be understood that two kinds of primers are preferably used from the viewpoint of improving arbitrariness of the base sequence of the information nucleic acid.

In the above step (1), it is particularly preferable that the blended solution is heated at 94° C. for 5 minutes. This is because DNA becomes difficult to be divided into two strands if the blended solution is heated at 92° C. for less than 2 minutes, and the enzyme is deactivated if the blended solution is heated at 95° C. for more than 5 minutes. It will be understood that the step (1) may not be needed in case that the information nucleic acid contained in the clear paint composition or the clear coat film is single-stranded one.

At the above heating (2a), it is particularly preferable that the blended solution is heated at 94° C. for 30 seconds. This is because a rate of amplification is lowered if the blended solution is heated at 92° C. for less than 30 seconds, and the enzyme is deactivated if the blended solution is heated at 95° C. for more than 60 seconds.

At the above heating (2b), it is particularly preferable that the blended solution is heated at 40° C. for 30 seconds. This is because the primer becomes difficult to bond to DNA if the blended solution is heated at 20° C. for less than 30 seconds, and the enzyme is deactivated if the blended solution is heated at 50° C. for more than 60 seconds.

At the above heating (2c), it is particularly preferable that the blended solution is heated at 72° C. for 30 seconds. This is because elongation of the information nucleic acid upon being amplified becomes insufficient if the blended solution is heated at 70° C. for less than 30 seconds, and the enzyme is deactivated if the blended solution is heated at 80° C. for more than 120 seconds.

In the above step (3), it is particularly preferable that the blended solution is heated at 72° C. for 7 minutes. This is because elongation of the information nucleic acid upon being amplified becomes unavoidably insufficient when the blended solution is heated at 70° C. for less than 1 minutes, and heating at 80° C. for more than 10 minutes is a waste of time.

Further, it is particularly preferable that repetition of the heat cycle consisting of the heatings (2a) to (2c) is 30 times or cycles. The rate of amplification is lowered if the repetition is less than 20 times, and the repetition of more than 50 times results in a waste of time.

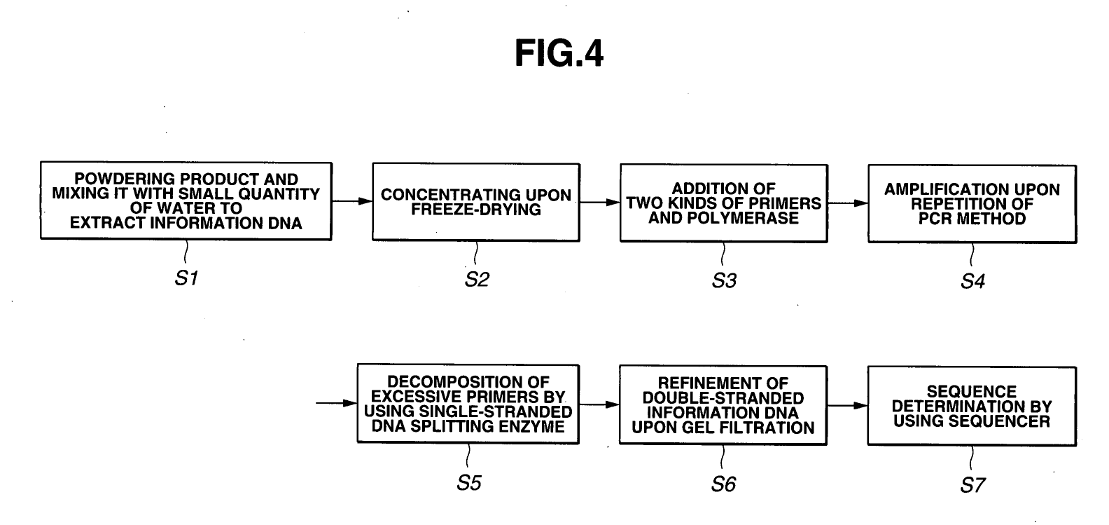

An example of the above individuality identification method is illustrated with reference to a flow chart of FIG. 4.

As shown in FIG. 4, the information DNA is extracted from the clear paint composition or the clear coat film in a step S1. The extracted information DNA is concentrated upon freeze-drying in a step S2. Two kinds of primers and a kind of polymerase are added to the concentrated information DNA in a step S3. DNA is amplified upon repetition of the PCR method in a step S4. An excessive primer is decomposed with a single-stranded DNA splitting enzyme in a step S5. The double-stranded information nucleic acid is refined upon gel filtration in a step S6. A sequence determination is carried out by using a sequencer in a step S7.

It will be understood that, for example, the clear coat film may be powdered and then mixed with a small quantity of water in the step S1: however, the information DNA is efficiently extracted by hydrolysis or the like, for example, in case that the information DNA is chemically bonded to the fine particles when carried on the fine particles. Additionally, in the step S2, concentrating may be carried out by using a centrifugal evaporator or the like. Further, in the step S5, Taq DNA polymerase, Tth DNA polymerase, Tfl DNA polymerase, Vent DNA polymerase, Pfu DNA polymerase, Bca BEST polymerase, KOD DNA polymerase and/or the like are used as the single-stranded DNA splitting enzyme. Further, repetition of cycle of the steps of S3 to S4 may be additionally inserted between the steps of S6 and S7 so as to amplify an objective DNA. In the step S7, a sequence determination using a mass spectroscope may be carried out so as to be combined with the sequence determination using the sequencer.

In the above individuality identification, it is preferable that the hydroxyl group at position 5′ is derivatized with biotin or fluorescent molecules from the viewpoints of improving the convenience in isolation and refinement of the information nucleic acid. Concretely, using biotin to derivatize a part of the information nucleic acid facilitates a selective adsorption of the information nucleic acid to a column to which avidin (a kind of protein) is bonded. Meanwhile, using fluorescent molecules such as fluorescein facilitates refinement and the like of the information nucleic acid since nucleic acid itself becomes fluorescent so as to be sensitively detectable.

Additionally, in such a case that the hydroxyl group at position 5′ of the information nucleic acid is substituted with sulfur, the information nucleic acid can be easily separated by being extracted with water and by being flown through a column containing a carrier coated with gold (Au).

### EXAMPLES

The present invention will be more readily understood with reference to the following Examples; however, these Examples are intended to illustrate the invention and are not to be construed to limit the scope of the invention.

(I) Preparation of Clear Paint containing Information Nucleic Acid having Primer Binding Site

First, an information DNA having a primer binding site was fixed on fine particles (i), (ii) and (iii) which will be discussed in (II) and were different from each other in size. Then, a certain amount (as shown in Table 1) of the fine particles (i), (ii) and (iii) were added upon being mixed to “Superlac 0-130 GN3” (the trade name of NIPPON PAINT CO., LTD.) under stirring followed by stirring for 1 hour thereby obtaining a clear paint of Examples 1 to 22 as shown in Table 1.

(II) Fine Particles used in (I)

(i) Zinc oxide available from SHOWA DENKO K.K. under the trade name of “ZS-032” and having an average particle size of 0.02 μm

(ii) Aluminium oxide available from MICRON Co., Ltd. under the trade name of “AW40-74” and having an average particle size of 40 μm

(iii) Aluminium oxide available from MICRON Co., Ltd. under the trade name of “AW50-74” and having an average particle size of 60 μm

(III) Forming of Laminated Coat Film

A cationic electrodeposition paint (a cationic electrodeposition paint available from NIPPON PAINT CO., LTD. under the trade name of “POWERTOP U600M”) was electrodeposition-coated on a zinc phosphate-treated dull steel plate 150 mm long by 70 mm wide by 0.8 mm thick so as to form a paint film having a thickness of 20 μm in a dried state. Then, the paint film was baked at 160° C. for 30 minutes. Thereafter, a gray intermediate coat (available from NOF Corporation under the trade name of “Hi-Epico No. 500”) was coated on the paint film so as to have a thickness of 30 μm and then baked at 140° C. for 30 minutes. Subsequently, the clear paint (“Superlac 0-130 GN3”) containing the information DNA was coated on the gray intermediate coat so as to have a thickness of 30 μm and then baked at 140° C. for 30 minutes.

Evaluations of detectable ability of DNA, moisture-resistance or adhesion, smoothness or apparatus, and discoloration resistance or transparency were conducted on the thus obtained laminated coat films of Examples 1 to 22.

[Evaluation of Detectable Ability of DNA]

The evaluation of detectable ability of DNA was conducted as follows:

(a) A test piece of the above information nucleic acid composition was finely fragmentized by using a cutter. (b) 5 mL of sterilized distilled water was added to the fragmentized test piece and then stirred by using a magnetic stirrer, thereby extracting DNA into a layer of water.

(c) The layer of water was separated from the fragmentized test piece by using a centrifugal separator and then concentrated in a centrifugal evaporator to obtain a concentrated DNA solution.

(d) The concentrated DNA solution (5 μL), PCR buffer (5 μL), Taq polymerase (0.25 μL), sterilized distilled water (24.75 μL), 5 μM of primer 1 (5 μL), 5 μM of primer 2 (5 μL) and 2 mM dNTP (5 μL) were mixed together to obtain a mixed solution. The primers 1 and 2 had the following base sequences:

Primer 1 5′-TGCACGCACCGTGTACTC-3′
Primer 2 5′-CCGACCAACGTGTCCACT-3′

(e) The mixed solution was heated at 94° C. for 5 minutes and then subjected to repetition of 30 cycles of a temperature control consisting of first heating at 94° C. for 30 seconds, second heating at 40° C. for 30 seconds and third heating at 72° C. for 30 seconds in the order mentioned.

(f) The mixed solution was heat-treated at 72° C. for 7 minutes and then preserved at 4° C.

(g) By using a single-stranded DNA splitting enzyme (S1 nuclease), excessive primers were split or decomposed. Thereafter, a gel filtration was carried out to remove the split primers so as to refine the objective double-stranded information DNA.

(h) 2,3-dideoxynucleosidetriphosphate provided with fluorescence and one kind of primer (the above primer 1) were mixed to the refined information DNA to obtain a mixture information DNA.

(i) The mixture information DNA was subjected to operations similar to those in the steps (d) to (f).

(j) The mixture information DNA was refined under the gel filtration and then supplied to an automatic sequencer to determine the base sequence of the information DNA.

Results of the evaluations under the above steps to try detection of the information DNA are given in Table 1 in which “A” indicates the fact that detecting and identifying the information DNA are easy; and “B” indicates the fact that additional PCR treatment is required to detect and identify the information DNA.

[Evaluation of Moisture-Resistance (or Adhesion)]

The laminated coat film was allowed to stand for 500 hours in an atmosphere having a temperature of 50° C. and a relative humidity of 95%. Thereafter, the laminated coat film was evaluated in adhesion according to the following process: First, 11 parallel and linear cuts at 2 mm intervals were made, and additionally 11 parallel and linear cuts at 2 mm intervals were made perpendicularly to the former 11 parallel and linear cuts so as to form 100 square cut grids, according to item 7.2 (2) (e) of JIS (Japanese Industrial Standard) K 5400. The linear cuts were made by using a cutter so as to reach the dull steel plate (the base material) on which the coat film was formed. Then, as regulated in JIS Z 1522, a tape was adhered onto the surface of the 100 square cut grids and then peeled off upward in a stroke. Thereafter, the number of the cut grids remaining not-peeled on the dull steel plate was counted. Results are given in Table 1.

[Evaluation of Smoothness (or Appearance)]

A condition of the surface of the laminated coat film was inspected by visual observation and judged according to standards in which “A” indicates the fact that the surface was generally smooth; “B” indicates the fact that the surface was slightly uneven; and “C” indicates the fact that the surface was uneven.

[Evaluation of Transparency]

The laminated coat film was allowed to stand for 500 hours in an atmosphere having a temperature of 50° C. and a relative humidity of 95%. Then, a degree of discoloration was evaluated by visual observation. As shown in Table 1, evaluation was made according to standards in which “A” indicates the fact that the laminated coat film was generally transparent; “B” indicates the fact that the laminated coat film was slightly whitish; and “C” indicates the fact that the laminated coat film was whitish.

TABLE 1

| | DNA content | Average | Content | Identification | | | |
|---|---|---|---|---|---|---|---|
| | (μg/100 g of | size of fine | of fine | of | Moisture- | | |
| | resinous | particles | particles | information | resistance | Smoothness | |
| Item | solid content) | (μm) | (%) | DNA | (Adhesion) | (Appearance) | Transparency |
| Example 1 | 0.5 | 0.02 | 0.5 | A | 100 | A | A |
| Example 2 | 0.5 | 0.02 | 10 | A | 100 | A | A |
| Example 3 | 0.5 | 2 | 0.5 | A | 100 | A | A |
| Example 4 | 0.5 | 2 | 1 | A | 100 | A | A |
| Example 5 | 100 | 0.02 | 0.5 | A | 100 | A | A |
| Example 6 | 100 | 0.02 | 10 | A | 100 | A | A |
| Example 7 | 100 | 2 | 0.5 | A | 100 | A | A |
| Example 8 | 100 | 2 | 1 | A | 100 | A | A |
| Example 9 | 500 | 0.02 | 0.5 | A | 100 | A | A |
| Example 10 | 500 | 0.02 | 10 | A | 100 | A | A |
| Example 11 | 500 | 2 | 0.5 | A | 100 | A | A |
| Example 12 | 500 | 2 | 1 | A | 100 | A | A |
| Example 13 | 0.1 | 0.02 | 0.5 | B | 100 | A | A |
| Example 14 | 0.1 | 0.02 | 10 | B | 100 | A | A |
| Example 15 | 0.1 | 2 | 0.5 | B | 100 | A | A |
| Example 16 | 0.1 | 2 | 10 | B | 100 | A | B |
| Example 17 | 0.5 | 0.02 | 0.2 | B | 100 | A | A |
| Example 18 | 0.5 | 0.02 | 20 | A | 90 | A | B |
| Example 19 | 0.5 | 4 | 0.2 | A | 100 | B | B |
| Example 20 | 0.5 | 4 | 10 | A | 80 | C | C |
| Example 21 | 1000 | 0.02 | 10 | A | 90 | A | B |
| Example 22 | 1000 | 2 | 10 | A | 70 | A | B |

As apparent from Table 1, in the clear coat films of Examples 1 to 12, the information DNA is excellently identified while good appearance and adhesion are exhibited. In other words, an intended appearance of the clear coat films can be obtained, and identification of the information DNA of the clear coat film becomes possible with the same workability as that of a usual coating process.

To the contrary, in the clear coat films of Examples 13 to 22, any of the detectable ability of the information DNA, the moisture-resistance, the smoothness and the transparency is found degraded since any of the content of the information DNA, the average size and the content of the fine particles is outside a preferable range in the present invention.

As appreciated from the above, according to the present invention, the objective products can be individually identified by determining the base sequence of the information nucleic acid contained therein, even if they are mass-produced products such as industrial products.

The entire contents of Japanese Patent Application P2004-362123 (filed Dec. 15, 2004) are incorporated herein by reference.

Although the invention has been described above by reference to certain embodiments and examples of the invention, the invention is not limited to the embodiments and examples described above. Modifications and variations of the embodiments and examples described above will occur to those skilled in the art, in light of the above teachings. The scope of the invention is defined with reference to the following claims.

What is claimed is:

1. A clear paint composition comprising:
a material of a clear paint; and
an information nucleic acid contained in the material of the clear paint and including a site having an arbitrary and known base sequence.

2. A clear paint composition as claimed in claim 1, wherein the information nucleic acid is contained in the material of the clear paint within a range of from 0.5 to 500 μg relative to 100 g of a resinous solid content of the clear paint.

3. A clear paint composition as claimed in claim 1, wherein the information nucleic acid is singly contained in the material of the clear paint and dispersed so as to avoid its secondary aggregation in water.

4. A clear paint composition as claimed in claim 1, further comprising fine particles on which the information nucleic acid is carried.

5. A clear paint composition as claimed in claim 4, wherein the fine particles have an average particle size ranging from 0.01 to 2 μm.

6. A clear paint composition as claimed in claim 4, wherein the fine particles is in a content ranging from 0.5 to 10% relative to a resinous solid content of the clear paint.

7. A clear paint composition as claimed in claim 1, wherein the material of the clear paint is of at least one property selected from the group consisting of lipophilic liquid, hydrophilic liquid and powder.

8. A clear coat film comprising:
a solidified clear paint composition including a material of a clear paint, and an information nucleic acid contained in the material of the clear paint and including a site having an arbitrary and known base sequence.

9. A clear coat film as claimed in claim 8, wherein the clear coat film is in one of a transparent and colorless state and a transparent and colored state.


SEQUENCE LISTING (2 sequences)

SEQ ID NO: 1 - DNA, 18 bp, Artificial Sequence
  Description of Artificial Sequence Synthetic primer
tgcacgcaccgtgtactc

SEQ ID NO: 2 - DNA, 18 bp, Artificial Sequence
  Description of Artificial Sequence Synthetic primer
ccgaccaacgtgtccact